(19) United States
(12) Patent Application Publication
(10) Pub. No.: US 2022/0095808 A1
(43) Pub. Date: Mar. 31, 2022

(54) THERAPEUTIC ORTHOPEDIC MATTRESS FOR PETS AND ASSOCIATED METHODS

(71) Applicant: Vanderlei GONCALVES, Orlando, FL (US)

(72) Inventor: Vanderlei GONCALVES, Orlando, FL (US)

(21) Appl. No.: 17/546,460

(22) Filed: Dec. 9, 2021

Related U.S. Application Data
(63) Continuation-in-part of application No. 15/853,340, filed on Dec. 22, 2017, now Pat. No. 11,202,516.
Application No. 15/853,340 is a continuation of application No. 15/622,332, filed on Jun. 14, 2017, now abandoned.
(60) Provisional application No. 62/334,535, filed on May 11, 2016.

(51) Int. Cl. A47C 27/14; A47C 31/00; A47C 27/15; A01K 1/035; A61N 2/06

(52) U.S. Cl. CPC A47C 27/144; A47C 31/004; A61N 2/06; A47C 27/15; A01K 1/0353; A47C 31/003

(57) ABSTRACT

A therapeutic orthopedic mattress for pets includes a top cover, a first layer covered by the top cover, and a second layer underlying the first layer. The mattress also includes an antennae comprising an elongated wire having a first end positioned proximate a first end of the first layer and a second end longitudinally spaced apart from the first end proximate a second opposing end of the first layer. The antennae is spread out between the first layer and the top cover. In addition, the method includes a microprocessor coupled to the first end of the antennae and is configured to distribute a plurality of sequences of intermittent electrical signals along the antennae to the second end. The mattress also includes a plurality of magnetic discs and far infrared radiation discs dispersed over a top surface of the first layer.

### RELATED APPLICATIONS

The present invention is a continuation-in-part of U.S. patent application Ser. No. 15/353,340 filed Dec. 22, 2017, now U.S. Pat. No. 11,202,516 issued Dec. 21, 2021 which is a continuation of U.S. patent application Ser. No 15/622,332, filed Jun. 14, 2017, now abandoned, which claims priority to Provisional Patent application Ser. No. 62/334,535 filed May 11, 2016, the entire contents of these applications incorporated herein by reference.

### TECHNICAL FIELD

The present invention relates to the field of mattresses, and, more particularly, to a therapeutic orthopedic mattress for pets and associated methods.

### BACKGROUND

Orthopedic mattresses have been developed for people who need extra support. Similarly, pets could also benefit from the extra support from an orthopedic mattress. Typical pet beds are placed on the floor and are of relatively simple construction of a fabric cover and a cushioning fill material inside. The fill material may be wood chips, fiber fill or foam chips, for example. Other types of pet beds use a layer of foam material.

A shortcoming of these types of beds are that they lack proper support for older pets that may have health issues, arthritis, or recovering from surgery and do not provide any therapeutic benefits. Accordingly, there is a need to develop an improved mattress that can provide proper support for a pet, and that can treat a physical condition and allow for restorative sleep.

### SUMMARY

A therapeutic orthopedic mattress for pets is disclosed. The mattress includes a top cover, a first layer having a first end and an opposing second end, where the first layer covered by the top cover. The mattress also includes a second layer underlying the first layer, and an antennae comprising an elongated wire having a first end positioned proximate the first end of the first layer and a second end longitudinally spaced apart from the first end proximate the second opposing end of the first layer. The antennae is spread out between the first layer and the top cover. In addition, the mattress includes a microprocessor coupled to the first end of the antennae and configured to distribute a plurality of sequences of intermittent electrical signals along the antennae to the second end. The plurality of sequences of intermittent electrical signals may comprise pulsed low-frequency electromagnetic waves, and the pulsed low-frequency electromagnetic waves may comprise different low frequencies and intensities.

The first layer may comprise natural latex foam, and the second layer may comprise a plurality of polyethylene interwoven fibers. In addition, the first layer may have a convoluted top surface with a plurality of peaks and valleys. The mattress may also include a plurality of magnetic discs dispersed over a top surface of the first layer, where each magnetic disc of the plurality of magnetic discs comprises a positive pole surface and an opposing negative pole surface with the negative pole surface facing towards the top layer. The mattress may include a plurality of far infrared radiation (FIR) discs dispersed over a top surface of the first layer.

In another aspect, a method of fabricating a therapeutic orthopedic mattress for a pet is disclosed. The method includes providing a first layer having a first end and an opposing second end and adhering a second layer under the first layer. The method also includes positioning an antennae comprising an elongated wire having a first end proximate the first end of the first layer and positioning a second end longitudinally spaced apart from the first end proximate the second opposing end of the first layer, where the antennae is spread out between the first layer and the top cover. The method also includes coupling a microprocessor to the first end of the antennae, where the microprocessor is configured to distribute a plurality of sequences of intermittent electrical signals along the antennae to the second end.

### BRIEF DESCRIPTION OF THE DRAWINGS

The aspects and the attendant advantages of the embodiments described herein will become more readily apparent by reference to the following detailed description when taken in conjunction with the accompanying drawings wherein:

FIG. 1 is a top view of a therapeutic orthopedic mattress for pets in which various aspects of the disclosure may be implemented;

FIG. 2 is an elevational view of the therapeutic orthopedic mattress of FIG. 1;

FIG. 3 is an exploded perspective view of the therapeutic orthopedic mattress of FIG. 1;

FIG. 4 is a cross sectional view of a first layer taken in the direction of line 4-4 of the therapeutic orthopedic mattress of FIG. 3;

FIG. 5 is a cross sectional view of a second layer taken in the direction of 5-5 of the therapeutic orthopedic mattress of FIG. 3; and

FIG. 6 is a top perspective view of a pulsed electro-magnetic field system carried by the first layer of the therapeutic orthopedic mattress of FIG. 1.

### DETAILED DESCRIPTION

The present invention will now be described more fully hereinafter with reference to the accompanying drawings, in which preferred embodiments of the invention are shown. This invention may, however, be embodied in many different forms and should not be construed as limited to the embodiments set forth herein. Rather, these embodiments are provided so that this disclosure will be thorough and complete, and will fully convey the scope of the invention to those skilled in the art. Like numbers refer to like elements throughout.

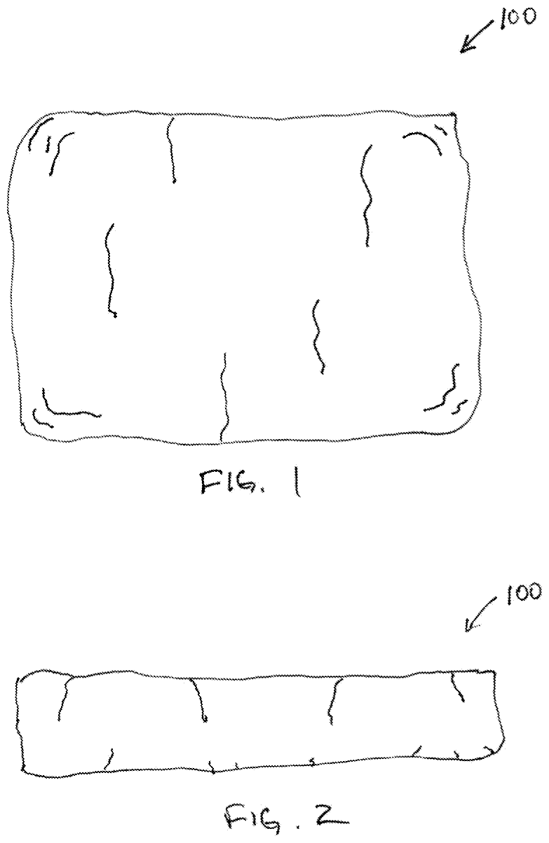

Referring now to FIGS. 1 and 2, a therapeutic orthopedic mattress for pets is shown and generally designated 100. The mattress 100 is directed to providing a resting place for pets such as dogs. The mattress 100 is placed on a floor or other relatively flat surface and has a generally rectangular shape. The size and dimensions of the mattress 100 may vary depending on the breed and size of the dog. In other aspects, the mattress 100 may be square or round, for example. In addition the thickness of the mattress 100 may also vary from one inch to three inches, for example.

The mattress 100 comprises a plurality of layers as best seen in FIG. 3. The mattress 100 includes a top cover 102. The top cover 102 comprises a preferably water resistant and durable material that is machine washable.

A first layer 104 of the mattress 100 comprises a natural latex material having a flat core convoluted cut design. The first layer 104 includes a system of bubbles interconnected with peaks and valleys 118, or egg crate style, with the peaks facing upwards. The convoluted cut design minimizes pressure points, which keeps the body of the pet in a natural temperature and maintaining the pet in a comfortable temperature. In addition, the convoluted cut design allows pockets of air around the pet and reduces heat by allowing airflow to move heat away from the pet and create a clean environment.

A second layer 106 of the mattress 100 comprises a plurality of polyethylene interwoven fibers. The second layer 106 provides full body support for the mattress 100 for years with little to no sag or pressure points. The plurality of polyethylene interwoven fibers of the second layer 106 comprise an interwoven nest-like structure that provides the ability for the second layer 106 to rebound back to and keep its original shape. Further, the second layer 106 includes a large percentage of empty space air between the plurality of interwoven fibers, which also promotes cooler temperatures when the pet is laying on the mattress 100.

Referring now to FIG. 4, a cross sectional view of the first layer 104 taken in the direction of line 4-4 of FIG. 3 is illustrated. The convoluted top surface 110 of the first layer 104 includes the peaks and valleys as discussed above. A bottom surface 112 of the first layer 104 is relatively flat. In a particular aspect, a plurality of magnetic discs 114 are dispersed over the top surface 110 of the first layer 104. Each magnetic disc 114 comprises a positive pole surface and an opposing negative pole surface with the negative pole surface facing towards the top cover 102.

The magnetic discs 114 may be, in a particular aspect, approximately ½ inch in diameter and ¼ inch in thickness with a round shape and generate a magnetic field of about 1500-2000 Gauss. The magnetic discs 114 preferably comprise ceramic ferrite. The magnetic discs 114 are placed in the valley locations of the convoluted top surface 110.

In another particular aspect, a plurality of far infrared radiation (FIR) discs 116 are dispersed over the convoluted top surface 110 of the first layer 104. Similar to the magnetic discs 114 discussed above, the FIR discs 116 are approximately ½ inch in diameter and ¼ inch in thickness with a round shape and emit a frequency having a wavelength of about 4μ-14μ. The FIR discs 116 may be placed in alternating valley locations with respect to the magnetic discs 114 on the convoluted top surface 110.

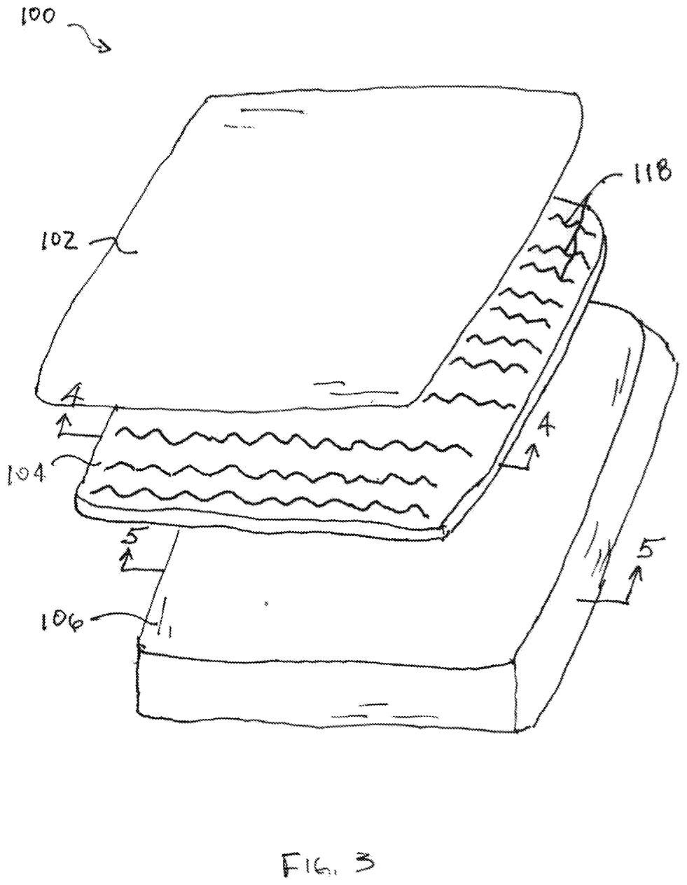

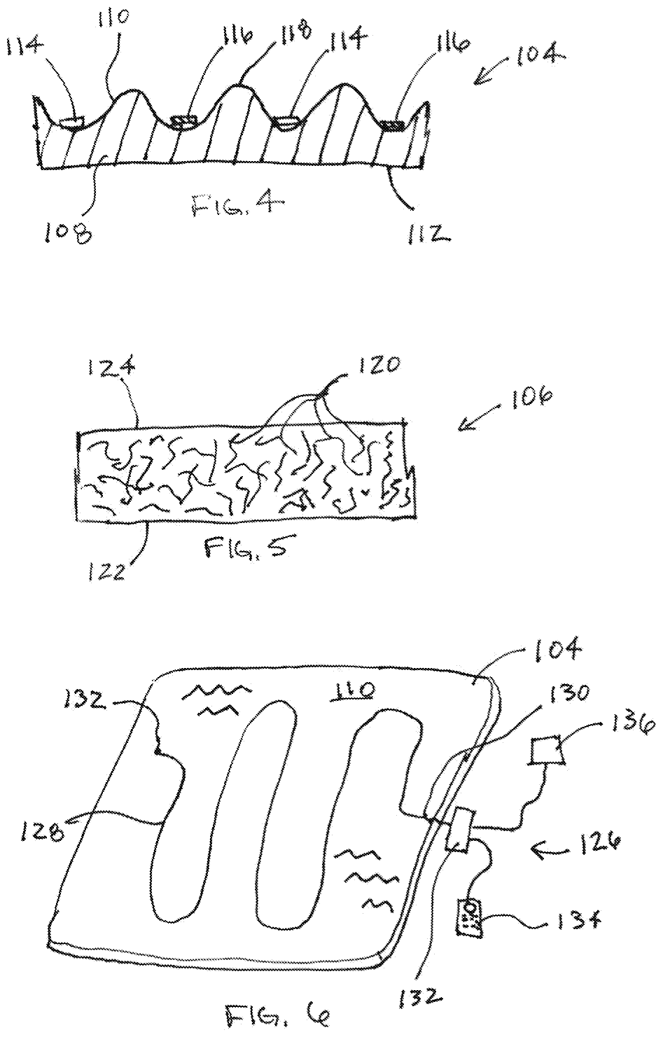

Referring now to FIG. 5, a cross sectional view of the second layer 106 taken in the direction of line 5-5 of FIG. 3 is illustrated. A top surface 124 of the second layer 106 is configured to mate to the bottom surface 112 of the first layer 104. The second layer 106 comprises a plurality of polyethylene interwoven fibers 120.

In a particular aspect, the mattress includes a pulsed electro-magnetic field (PEMF) system 126 as illustrated in FIG. 6. The PEMF system 126 includes a microprocessor 132 having an antennae 128 or coils positioned between the first layer 104 and the top cover 102 of the mattress 100. The microprocessor 132 is positioned outside the mattress 100 so that it is easy to access and repair if needed.

The PEMF system 126 emits low-frequency pulsed electromagnetic waves through the top cover 102 of the mattress 100. The pulsed electromagnetic waves with different frequencies and intensities are configured to reach the acupuncture points of the pet to induce restoring energy, relax the nerves, reduce stress, and encourage relaxation to sleep.

The antennae 128 may comprise an elongated wire hand have a first end 130 positioned proximate the first end of the first layer 104 and a second end 132 longitudinally spaced apart from the first, end 104 and proximate the second opposing end of the first layer 104. The antennae 128 is spread out on the top surface 110 of the first layer 104 so that it is between the first layer 104 and the top cover 102.

The microprocessor 132 is coupled to the first end 130 of the antennae 128 and is configured to distribute a plurality of sequences of intermittent electrical signals along the antennae 128 to the second end 132. In addition, a controller 134 and a power source 136 are coupled to the microprocessor 132.

In another aspect, a method of fabricating a therapeutic orthopedic mattress for a pet includes providing the first layer 104 having a first end and an opposing second end and adhering the second layer 106 under the first layer 104. The method also includes positioning the first end 130 of the antennae 128 proximate the first end of the first layer 104 and positioning the second end 132 longitudinally spaced apart from the first end proximate the second opposing end of the first layer 104. In addition, the method includes coupling the microprocessor 132 to the first end 130 of the antennae 128, where the microprocessor 132 is configured to distribute a plurality of sequences of intermittent electrical signals along the antennae 128 to the second end 132. The method may also include positioning the plurality of magnetic discs 114 over the top surface of the first layer 104, positioning the plurality of far infrared radiation (FIR) discs 116 over the top surface of the first layer 104, and placing the top cover 102 over the first layer 104.

Many modifications and other embodiments of the invention will come to the mind of one skilled in the art having the benefit of the teachings presented in the foregoing descriptions and the associated drawings. Therefore, it is understood that the invention is not to be limited to the specific embodiments disclosed, and that modifications and embodiments are intended to be included within the scope of the appended claims.

That which is claimed is:

1. A therapeutic orthopedic mattress for pets comprising:
a top cover;
a first layer having a first end and an opposing second end, the first layer covered by the top cover;
a second layer underlying the first layer;
an antennae comprising an elongated wire having a first end positioned proximate the first end of the first layer and a second end longitudinally spaced apart from the first end proximate the second opposing end of the first layer, the antennae spread out between the first layer and the top cover; and
a microprocessor coupled to the first end of the antennae and configured to distribute a plurality of sequences of intermittent electrical signals along the antennae to the second end.

2. The therapeutic orthopedic mattress of claim 1, wherein the first layer comprises natural latex foam.

3. The therapeutic orthopedic mattress of claim 1, wherein the second layer comprises a plurality of polyethylene interwoven fibers.

4. The therapeutic orthopedic mattress of claim 1, wherein the first layer comprises a convoluted top surface having a plurality of peaks and valleys.

5. The therapeutic orthopedic mattress of claim 1, further comprising a plurality of magnetic discs dispersed over a top surface of the first layer.

6. The therapeutic orthopedic mattress of claim 5, wherein each magnet discs of the plurality of magnetic discs comprises a positive pole surface and an opposing negative pole surface with the negative pole surface facing towards the top cover.

7. The therapeutic orthopedic mattress of claim 1, further comprising a plurality of far infrared radiation (FIR) discs dispersed over a top surface of the first layer.

8. The therapeutic orthopedic mattress of claim 1, wherein the plurality of sequences of intermittent electrical signals comprise pulsed low-frequency electromagnetic waves.

9. The therapeutic orthopedic mattress of claim 8, wherein the pulsed low-frequency electromagnetic waves comprise different low frequencies and intensities.

10. The therapeutic orthopedic mattress of claim 1, further comprising a controller and a power source coupled to the microprocessor.

11. A therapeutic orthopedic mattress for pets comprising:
a top cover;
a first layer having a first end and an opposing second end, the first layer covered by the top cover; and
an antennae comprising an elongated wire having a first end positioned proximate the first end of the first layer and a second end longitudinally spaced apart from the first end proximate the second opposing end of the first layer, the antennae spread out between the first layer and the top cover.

12. The therapeutic orthopedic mattress of claim 11, further comprising a second layer underlying the first layer.

13. The therapeutic orthopedic mattress of claim 11, further comprising a microprocessor coupled to the first end of the antennae and configured to distribute a plurality of sequences of intermittent electrical signals along the antennae to the second end.

14. The therapeutic orthopedic mattress of claim 11, wherein the first layer comprises natural latex foam.

15. The therapeutic orthopedic mattress of claim 11, wherein the second layer comprises a plurality of polyethylene interwoven fibers.

16. The therapeutic orthopedic mattress of claim 11, wherein the first layer comprises a convoluted top surface having a plurality of peaks and valleys.

17. The therapeutic orthopedic mattress of claim 11, further comprising a plurality of magnetic discs dispersed over a top surface of the first layer.

18. The therapeutic orthopedic mattress of claim 11, further comprising a plurality of far infrared radiation (FIR) discs dispersed over a top surface of the first layer.

19. A method of fabricating a therapeutic orthopedic mattress for a pet, the method comprising:
providing a first layer having a first end and an opposing second end;
adhering a second layer under the first layer;
positioning an antennae comprising an elongated wire having a first end proximate the first end of the first layer and positioning a second end longitudinally spaced apart from the first end proximate the second opposing end of the first layer, wherein the antennae is spread out between the first layer and the top cover; and
coupling a microprocessor to the first end of the antennae, wherein the microprocessor is configured to distribute a plurality of sequences of intermittent electrical signals along the antennae to the second end.

20. The method of claim 19, farther comprising:
positioning a plurality of magnetic discs over a top surface of the first layer;
positioning a plurality of far infrared radiation (FIR) discs over a top surface of the first layer; and
placing a top cover over the first layer.
